(19) United States
(12) Patent Application Publication
(10) Pub. No.: US 2009/0285869 A1
(43) Pub. Date: Nov. 19, 2009

(54) SALT STABLE LECITHIN ORGANOGEL COMPOSITION

(75) Inventor: John Olin Trimble, Texarkana, TX (US)

Correspondence Address: JACKSON WALKER LLP, 901 MAIN STREET, SUITE 6000, DALLAS, TX, 75202-3797, US

(73) Assignee: Humco Holding Group, Inc., Texarkana, TX (US)

(21) Appl. No.: 12/465,171

(22) Filed: May 13, 2009

Related U.S. Application Data
(60) Provisional application No. 61/127,651, filed on May 14, 2008.

(51) Int. Cl. A61K 8/06; A61K 8/92; A61K 8/55

(52) U.S. Cl. 424/401; 514/784

(57) ABSTRACT

A lecithin organogel (“LO”) operates as a transdermal pharmaceutical delivery composition. In particular, the lecithin organogel comprises an internal oil phase containing oil-in-water (“O/W”) and water-in-oil (“W/O”) emulsifiers, and an aqueous phase comprising inorganic and organic hydrocolloids. The lecithin organogel may contain up to 80% additive ingredients, including biocompatible surfactants, nonpolar solvents, saturated fatty alcohols, moisturizers, preservatives or antimicrobials, and chelating agents.

The present invention claims priority to U.S. Provisional Patent Application Ser. No. 61/127,651 filed May 14, 2008, the entire content of which is hereby incorporated by reference.

### BACKGROUND

The present invention relates to a transdermal pharmaceutical delivery composition, including matrices of a lecithin gel, such as a lecithin organogel (“LO”). In particular, this invention relates to compositions which comprise an internal oil phase containing oil-in-water (“O/W”) and water-in-oil (“W/O”) emulsifiers, and an aqueous phase comprising inorganic and organic hydrocolloids. Microscopically, these compositions maintain emulsion droplet integrity while macroscopically there is little or no viscosity decrease.

Topical agents have had relatively poor bioavailability in the past. With the advent of LO the problem of bioavailability has been somewhat resolved. It provides an adequate vehicle that permeates the stratum corneum. Lecithin is able to pass through the stratum corneum because it is a lipophilic substance. Both a drug and a hydrophobic medium can pass through the epidermis when the water-soluble drug is added to the hydrophobic substance. Bioavailability may range from about 10% to 60%.

Several different compositions for LO have been described. U.S. Pat. No. 5,654,337 to Roentsch, et al., issued Aug. 5, 1997, relates to a composition useful in the delivery of pharmaceutically active agents through the skin. The composition is formulated with a non-steroidal anti-inflammatory agent, such as ibuprofen or ketoprofen. Such formulation is rapidly absorbed through the skin to provide local relief from pain. In another embodiment of the invention, the composition is formulated with an antineoplastic agent. Such formulation is rapidly absorbed through the skin to provide local delivery to subcutaneous tumors. The composition is useful for transcutaneous delivery of other pharmaceutically-active compounds.

U.S. Pat. No. 5,716,639 to Carlsson, et al., issued Feb. 10, 1998, relates to a lipophilic carrier preparation having a continuous lipid phase, comprising a polar lipid material in combination with a non-polar lipid, and optionally a polar solvent, wherein the polar lipid material is a galacto-lipid material. The patent also describes the use of said lipophilic carrier preparation as a carrier for an active substance in a pharmaceutical composition, but also in nutritional, cosmetical and food products, as well as to a pharmaceutical composition. The invention relates to a lipophilic carrier preparation having a continuous lipid phase, comprising a polar lipid material in combination with a non-polar lipid, and optionally a polar solvent, wherein the polar lipid material is a galactolipid material. The invention also relates to the use of said lipophilic carrier preparation as a carrier for an active substance in a pharmaceutical composition, but also in nutritional, cosmetic and food products, as well as to a pharmaceutical composition.

U.S. Pat. No. 5,837,289 to Grasela, et al., issued Nov. 17, 1998, describes a composition and procedures for its formation and administration, which provide for a convenient, efficacious and simple transdermal administration of medications from a topically applied cream. No transmission through a membrane is involved. The composition incorporates at least two separate penetration enhancers which function synergistically to provide for rapid but controllable transport of the medication from the cream into the skin. The use of a plurality of penetration enhancers, at least one of which facilitates the separation of medication from the cream and at least a second of which alters the structure of the outer layers of skin, particularly the stratum corneum, enhances migration of the drug through the stratum corneum.

U.S. Pat. No. 6,290,986 to Murdock, et al., issued Sep. 18, 2001, provides a method and composition for transdermal delivery of pharmaceuticals or combinations of pharmaceuticals. The pharmaceuticals are delivered using a matrix of a lecithin gel such as a lecithin organogel. A number of psychopharmaceuticals can be used including fluoxetine, sertraline, carbamazepine, paroxetine, amitriptyline, trazadone, venlafaxine, propranolol, buproprion, valproic acid, nefazodone, ketoprofen, gabapentin, piroxican, doxepin, guaifenesin, pemoline and doxepin and combinations.

There are some commercial sources of LO. Gallipot, Inc. (St. Paul, Minn.) manufactures Lipobase®. This product is a water removable oil-in-water emulsion base containing natural oils and liposomes in a gel matrix. It does not contain petrolatum or mineral oil. It is fragrance, dye, and paraben free.

J.A.R. Pharmaceuticals, Ltd. (Edmonton, Alberta, Canada) manufactures Phlojel® Ultra. It is a lecithin organogel that has been formulated to have cosmetic properties, being non-greasy and having improved skin penetration of incorporated active ingredients. After application to the skin, it rapidly disappears leaving no residue. It has been widely accepted as a superior vehicle for delivering drugs across the skin barrier where relatively high local concentrations of drug are obtained from small applied doses. It also allows drug to perfuse the skin reaching the general circulation when desired, making the topical route an important alternative to the oral route which often is intolerant to the drug.

Medisca, Inc. (Plattsburgh, N.Y.) manufactures Lipo Cream Base.

Professional Compounding Centers of America (“PCCA”) (Houston, Tex.) manufactures Lipoderm®. It contains a patented liposomal component that provides the system with a Chemical Penetration Enhancer value comparable to a pluronic lecithin organogel and speed gel. It allows the medication to reach the system's circulation and local areas with efficiency. It is a stable system, which does not separate upon refrigeration or in the presence of ionic substances.

Almost all of the above matrices of LO achieve some tissue-levels of active compounds, reducing blood-level related side effects. Many of the commercial matrices of LO do not remain uniform and usable with the addition of up to 35% by weight total of ionic materials. They do not satisfy the demand of varying active compounds, as well as pharmacists' demands for time-efficient and uniform compounding, and patients' demands for cosmetic and efficacious prescriptions.

### SUMMARY

The present invention relates to a transdermal pharmaceutical delivery composition, including matrices of a lecithin gel such as a lecithin organogel. In particular, this invention relates to compositions which may comprise an internal oil phase containing optional oil-in-water (“O/W”) and water-in-oil (“W/O”) emulsifiers, and an aqueous phase comprising inorganic and organic hydrocolloids.

The current invention comprises a lecithin organogel composition which could be used to deliver pharmaceutical products transdermally. The invention further comprises a method for producing the lecithin organogel composition, which may contain up to 80% additive ingredients.

Lecithin organogel (“LO”) is a transdermal carrier used by pharmacists to deliver drugs through the skin when other routes of administration are not viable. It is non-irritating to the skin and is absorbed quickly. It is best used with drugs with molecular weights preferably less than about 400. It may include emulsifiers, hydrocolloids, isopropyl palmitate, lecithin, oil, and water.

Emulsifiers are used in creams and lotions to mix water with oils. They are necessary to form a homogenous mixture keeping water and oil together since water and oil do not mix but stay separated. There are 2 types of emulsifiers. Oil-in-water (“O/W”) emulsifiers keep oil drops packed in water, and are used more in moisturizing products. Water-in-oil (“W/O”) emulsifiers keep water drops packed in oil, and are used for a fatty feel.

Hydrocolloids are defined as colloid systems wherein the colloid particles are dispersed in water. They have colloid particles spread throughout water and can take on a gel state.

Lecithin is a naturally occurring mixture of diglycerides of fatty acids linked to the choline ester of phosphoric acid. It is used as a penetration enhancer in compounding LO. It is a liquid at room temperature and may become solid upon cooling. It is normally stored at room temperature. Lecithins vary greatly in their physical form from semiliquids to powders. They are almost odorless and vary from brown to light yellow. They decompose at extreme pH and are hygroscopic. They will oxidize and darken at high temperatures. Lecithin is usually stored at room temperature and protected from light. Refrigeration may cause the material to separate.

Isopropyl palmitate is a non-oleaginous emollient with a high capacity for spreading.

To form LO, lecithin is added to isopropyl palmitate and mixed until smooth. A sufficient amount of emulsifiers in oil is incorporated. A sufficient amount of hydrocolloids in water is incorporated.

Previously, the addition of greater than 15% to 20% of ionic materials to LO would result in marked viscosity decrease of the gel. The current invention allows the addition of up to 35% by weight total of ionic materials. Examples of ionic materials are amitriptyline, benzocaine, cyclobenzaprine, gabapentin, ketamine, lidocaine, methimazole, prilocaine, promethazine, and tetracaine.

In order to understand how LO can be used to deliver materials transdermally, it is important to first understand the barriers in the skin which prevent absorption into the skin. The skin is composed of three major components: the epidermis, the dermis, and the underlying subdermal tissue. The epidermis is composed of five different layers: stratum corneum; stratum lucidum; stratum granulosum; stratum spinosum; stratum basale. The stratum corneum is the most impermeable of these five layers. The stratum corneum can be compared to a brick wall. The stratum corneum consists of flattened cells imbedded in a lipid intercellular matrix just as a brick wall consists of bricks and mortar.

Without wishing to be bound by theory, two mechanisms for gel permeation into the skin have been proposed. One possible mechanism for gel permeation into the skin occurs by diffusion through the lipid intercellular matrix described above. Another proposed mechanism is that LO provides a slight disorganization of the skin allowing permeation of the gel and the active drug through the statum corneum. One thing that seems clear is that the lecithin component of LO has the ability to act as an amphoteric surfactant and enables many drugs to penetrate the dermal layer.

Emulsifiers can be classified according to their differing proportions of lipophilic and hydrophilic molecular structures. This ratio is characterized by the hydrophilic-lipophilic balance (“HLB”). It is a measure of the water affinity or oil affinity of the emulsifier. Emulsifiers are classified on a scale from 0 to 20. Those with HLB values of 8-18 are hydrophilic emulsifiers that exhibit higher water solubility and tend to form O/W emulsions. Those with HLB values of 1-8 are more lipophilic molecules that are more inclined to solubilize in the oil phase and thus tend to form W/O emulsions.

Non-ionic emulsifiers typically have good skin compatibility. Improved sensoric properties are obtained when combining these O/W and W/O emulsifiers.

Non-ionic O/W emulsifiers incorporate water-soluble ingredients into the oil phase. They may comprise addition products of 2 to 50 moles of ethylene oxide to linear fatty alcohols having 8 to 40 carbon atoms. O/W emulsifiers based on cetyl (C16), cetearyl (C16-18), and stearyl (C18) alcohol are excellent emulsifiers for cosmetic creams and lotions. O/W emulsifiers based on oleyl alcohol (C18:1) are more often used for the formulation of microemulsion systems. The HLB of these emulsifiers corresponds to the degree of ethoxylation. Approximate HLB values in ascending order are: Oleth-5 (9.0), Ceteareth-6 (10.0), Oleth-10 (12.4), Steareth-10 (12.4), Ceteareth-12 (13.4), Steareth-20 (15.3), Oleth-20 (15.5), Ceteth-20 (15.7), Ceteareth-20 (15.7), and Ceteareth-25 (16.2).

Non-ionic W/O emulsifiers incorporate oil-soluble ingredients into the water phase. They may comprise glycerol esters of alkanecarboxylic acids with a chain length of from 8 to 24 carbon atoms. W/O emulsifiers based on ricinoleic (C18), oleic (C18), stearic (C18), and lauric (C12) acid are excellent emulsifiers for cosmetic creams and lotions. The HLB of these emulsifiers corresponds to the length of carbon chain. Approximate HLB values in ascending order are: Glyceryl Ricinoleate (2.6), Glyceryl Oleate (2.8), Glyceryl Stearate (4.0), Glyceryl Dilaurate (4.0), and Glyceryl Laurate (5.2).

Inorganic hydrocolloids may comprise natural and synthetic bentonites, hectorites, and hydrotalcites.

Natural bentonite clay is magnesium aluminum silicate. It is used for thickening aqueous systems. Synthetic bentonite clay has positively charged organic material linked to the clay negative surface through cation exchange. It is used for thickening solvent systems.

Synthetic hectorite clay is sodium magnesium silicate. It is a rheology modifier with cation exchangeable clay used to improve pseudoplastic properties. It is used in hydrophobic systems to link organic materials.

Synthetic hydrotalcite clay is also magnesium aluminum silicate. It is reverse bentonite clay with negatively charged organic material linked to the clay positive surface through anion exchange. It is used in hydrophilic systems to encapsulate ionic materials.

Organic hydrocolloids may comprise natural and synthetic gums, polymers, and starches.

Natural gums are most often grouped together by their sources. Most are considered natural products. The most widely used of the plant exudates are gum arabic, gum tragacanth, gum karaya, and gum ghatti. Plant extracts are agar-agar, alginates, carrageenan, konjac and pectin. Guar and locust bean gum are seed gums. Microbial polysaccharides produced as microbial exudates include xanthan gum, curdlan and gellan gum. Cellulosics are microcrystalline cellulose, methylcellulose and hydroxypropyl methylcellulose. Animal sources are gelatin and chitosan.

Synthetic polymers suspend and stabilize the appearance of emulsifier-based products. Acrylate polymers are effective over a wide pH range from 3.5 to 10.0. They function synergistically with salts and emulsifiers providing options for achieving various suspending and stabilizing requirements.

Synthetic starches have a number of substituent groups reacted onto the hydroxyl groups of the polyglucose starch backbone to modify behavior of starches. Hydrophobic moieties can be attached to alter solubility and to enhance affinity for oil. The reaction of cross-linker with starch can increase starch viscosity stability and its tolerance to pH. Hydroxypropylation can provide greater aqueous stability and improved solubility.

Lecithin Organogel (“LO”) may be prepared by mixing an oil phase with inorganic and organic hydrocolloid phases using a high-shear mixing method. To prepare the oil phase, the kettle is charged with Lipowax® D (Lipo Chemicals Inc., Patterson, N.J.), Cetyl Alcohol, Stearyl Alcohol, Emulsynt® GDL (ISP, Wayne, N.J.), and Wheat Germ Oil, the mixer is installed, the temperature is raised to 75°-80° C., and the mixer is turned on. Next is the preparation of the inorganic hydrocolloid phase. In a tank equipped with a mixer, Purified Water is added, then the mixer is turned on, the temperature is raised to 35°-40° C., Veegum® HS (RT Vanderbilt Company, Norwalk, Conn.) is added, and mixing is carried out until dissolved. Next is the organic hydrocolloid phase. In a tank equipped with a mixer, Purified Water is added, then the mixer is turned on and heated to 35°-40° C. Structure® XL (National Starch, Bridgewater, N.J.) is added and mixed until dissolved. For the emulsion phase, the Oil Phase, the Inorganic Hydrocolloid Phase, and the Organic Hydrocolloid Phase are combined in a kettle with mixing. With mixing, Lecithin 33% Solution, Euxyl® PE9010 (Schulke & Mayr, Norderstedt, Germany), and Dow Corning (Midland, Mich.) 200-350 are added and mixed for 1 hour, then the mixture is transferred to a storage vat.

Lecithin organogel (LO) is composed of emulsifiers, hydrocolloids, and lecithin. It is a second generation pluronic lecithin organogel (“PLO”). The pluronic component has been removed. The advantages of the LO compared to the original PLO are that it is non-greasy, non-tacky, has improved stability to temperature, and has improved stability to salts.

The emulsion phase may be prepared by adding oil-in-water (O/W) and water-in-oil (W/O) emulsifiers to oil and agitating periodically to ensure complete dissolution. O/W Lipowax® D (Lipo Chemicals Inc., Patterson, N.J.) (cetearyl alcohol and ceteareth-20) is an emollient, thickener, and emulsion stabilizer. It is derived from naturally occurring fatty acids from coconut oil. W/O Emulsynt® GDL (ISP, Wayne, N.J.) (glyceryl dilaurate) is also an emollient, thickener, and emulsion stabilizer. It is produced from glycerin and lauric acid.

The hydrocolloid phase may be prepared by adding inorganic and organic hydrocolloids to water and agitating periodically to ensure complete dissolution. Inorganic Veegum® HS (RT Vanderbilt Company, Norwalk, Conn.) (magnesium aluminum silicate) is naturally occurring water-washed smectite clay used worldwide to stabilize emulsions and suspensions and to thicken a wide range of products. It provides maximum electrolyte stability and minimum acid demand. It is used in acid pH pharmaceutical suspensions. Organic Structure® XL (National Starch, Bridgewater, N.J.) (hydroxypropyl starch phosphate) is a modified starch which aids in emulsion stabilization and viscosity build. Emulsions containing it have outstanding stability over a broad temperature range of −30° C. up to 50° C. It also brings body to formulations and a conditioning after feel.

The lecithin phase may be prepared by mixing lecithin and isopropyl palmitate and allowing the mixture to stand overnight to ensure complete dissolution. The role of organic solvent in providing the desired solvent action onto the lecithin molecules is much emphasized. A large variety of organic solvents are able to form gel in the presence of lecithin. Isopropyl palmitate is of particular interest for topical applications of lecithin organogels. This has been attributed to its skin penetration enhancing property as well as its biocompatible and biodegradable nature.

Dispersion of a hydrophilic drug in the aqueous phase is conducted by dissolving the drug into lecithin organogel (LO). Hydrophilic drugs include the cancer and aids drug, promethazine; the diabetes and wound treatment drugs, amitriptyline and gabapentin; the pain and sports drugs, benzocaine, cyclobenzaprine, ketamine, lidocaine, prilocaine, and tetracaine; the veterinary drug, methimazole. Hydrophilic drugs have an uptake capacity of about 30% to about 35%.

Dispersion of a lipophilic drug in the oil phase is conducted by dissolving the drug into lecithin organogel (LO). Lipophilic drugs include the cancer and aids drug, haloperidol; the pain and sports drugs, ibuprofen, ketoprofen, and piroxicam. Lipophilic drugs have an uptake capacity of about 5% to about 10%.

Pluronic Lecithin Organogel (“PLO”) must use a levigating agent to wet substances and then incorporate the wetted substance into the base. LO provides opportunities for direct incorporation of a wide range of substances with diverse physicochemical characters of chemical nature, solubility, molecular weight, and size. It has generated considerable interest over the years as a potential topical drug delivery vehicle. The coexistence of organic and aqueous phase by means of a structurally well-defined micellar network of phospholipids and a large interfacial area makes it useful for a variety of applications. The topical applications of various drugs containing LO systems have been demonstrated to significantly enhance the skin permeation and absorption of both lipophilic and hydrophilic substances. The organized microstructural matrix and amphiphilicity of the biolipids with skin tissues are some of the major promoting factors for an enhanced transport of drug molecules into or across the skin. Therapeutic compounds of different chemical and physicochemical background have been incorporated in LO with some very encouraging results (Lawrence M. J., 2000).

Pluronic Lecithin Organogel (“PLO”) must be placed in a refrigerator for 24-48 hours to form a clear solution. Spontaneous LO formation by virtue of self-assembled supramolecular arrangement of surfactant molecules makes processing very simple and easy to handle. The incorporation of emulsifiers and hydrocolloids in LO is useful as cosurfactants and stablizers. This inclusion makes the organogelling feasible with lecithin of relatively lesser purity (Crandall W. T., 2001).

Pluronic Lecithin Organogel (“PLO”) must be stored at room temperature and has a shelf life of approximately twelve months. The structural integrity of LO is maintained for wider temperature ranges and longer time periods. The phase behavior of organogels varies on changing temperature conditions. The phase transition temperature (“PTT”) of sol-to-gel or gel-to-sol gives an insight into the nature of microstructures that form the gelling cross-linked network. The phase transition temperatures also help in optimizing the organogel composition. Concentration of emulsifiers and hydrocolloids in a given LO formulation are optimized by monitoring the PTTs of the organogel. PTT also reveals the microstructural homogeneity of the prepared organogel system. A narrow PTT range is indicative of homogenous microstructures within the gel (Jibry N., 2004).

Pluronic Lecithin Organogel (“PLO”) must avoid various ionic salts, surfactants, polymers, and cosolvents which have marked effects on micellization and solubilization. The solubility of various ionic drugs is increased in LO compared to PLO. The balanced hydrophilic and lipophilic character of LO also efficiently partitions with skin and therefore enhances skin penetration and transport of molecules. Significantly enhanced permeability of micellar-borne ionic drugs across human skin is observed when employing ionic drugs in LO compared to PLO. This permeation enhancing effect of LO is attributed to vectoring properties of reverse micelles. These micellar entities being small in size and with hydrocarbon sheath are received by the skin barrier as hydrophobic entities. This allows for closer interaction with the skin barrier leading to enhanced permeation of drug molecules (Willimann H., 1992).

Pluronic Lecithin Organogel (“PLO”) must have a greasy and tacky feel. LO provides non-greasy and non-tacky hydration of skin in a lipid-enriched environment so as to maintain the bioactive state of skin. Non-ionic emulsifiers facilitate penetration of both hydrophilic and hydrophobic drugs. Their low melting point of 30° C. results in fluidization of micelle bilayers following formula application. They also impart a lasting emollient feel (Brucks R., 1998).

A wide range of Pluronic Lecithin Organogel (“PLO”) is commercially available. Antiemetics include dexamethasone, dimenhydrate, and scopolamine. Muscle relaxants include cyclobenzaprine, baclofen, and buspirone. Neuropathy drugs include clonidine, capsaicin, and phenytoin. Pain management drugs include diclofenac, ibuprofen, ketoprofen, and indomethacin. Systemic analgesics include acetaminophen, hydromorphone, and morphine sulfate. Systemic hormones include progesterone and testosterone.

In addition to the range of commercially available Pluronic Lecithin Organogel (“PLO”), an even wider range of LO is now commercially available. Additional antiemetics include promethazine. Additional neuropathy drugs include amitriptyline and gabapentin. Additional pain management drugs include benzocaine, cyclobenzaprine, ketamine, lidocaine, prilocaine, and tetracaine. Additional veterinary drugs include methimazole.

Dermal and transdermal delivery have quickly gained acceptance as a unique delivery route providing an alternative to existing oral therapeutic regimens. The skin is a virtually impermeable barrier to most environmental and synthetic compounds. Hydrophobic and now hydrophilic compounds may permeate the skin to deliver therapeutically relevant amounts of active drugs. Some therapeutic advantages to dermal and transdermal delivery include: avoidance of local gastrointestinal toxicities; avoidance of first-pass metabolism; concentration of drug at localized sites where it is needed.

Dermal delivery can be a superior alternative for active drugs which are potent. Enhanced dermal skin penetration and site-specific delivery of hydrophilic actives into the deeper layers of skin has been achieved employing LO as a topical vehicle. There is an advantage in dermal delivery for active drugs which exert their action locally because of the high incidence of side-effects after oral administration which is directly correlated with blood concentrations. Low concentrations of potent active drugs in the bloodstream likewise minimize side effects.

The absolute bioavilability of a compound delivered transdermally is generally less than that delivered orally unless the compound is highly metabolized in the liver. LO has been used as a matrix for this transdermal transport of different hydrophobic compounds. Preliminary studies have indicated that the bioavailability of active drugs applied topically is approximately 10% to 60% of an equivalent oral dose.

A number of thickening and stabilizing agents are available for use in transdermal delivery systems. Poloxamer 407 is used as a co-emulsifier and consistency enhancer in Pluronic Lecithin Organogel (“PLO”). The addition of ionic materials reduces the gel formation temperature as well as the viscosity and pour point.

Non-ionic water-in-oil (“W/O”) emulsifiers such glyceryl dilaurate may be used as emulsifiers for creams and lotions that are rich in non-polar oils and waxes, and will facilitate the addition of ionic materials. They are very useful for increasing the body and stability, and improving the elegance of cost effective formulations and low pH formulations. W/O emulsifiers also have co-emulsifying characteristics, can couple more lipophilic materials, and promote emulsion stability. They deliver a very rich, silky, non-greasy after feel. Finally, W/O emulsifiers provide excellent spreadability and reduce product drag during application.

Non-ionic oil-in-water (O/W) emulsifiers such as ceteareth-20 may be used as emulsifiers for creams and lotions that are rich in polar oils and waxes, and will facilitate the addition of ionic materials. They are ethylene oxide adducts of high molecular weight saturated fatty alcohols designed to provide optimum emulsification and consistency. O/W emulsifiers are also compatible with anionic, cationic, and non-ionic surfactants, have good electrolyte tolerance, and are stable over a wide pH range.

Organic hydrocolloids such as hydroxypropyl starch phosphate aid in emulsion stabilization, aesthetics enhancement, and viscosity build. They have outstanding stability over a broad temperature range of −30° C up to 50° C. Organic hydrocolloids also bring body and a conditioning after feel. They provide flexibility over a wide pH range with high amounts of mono- and divalent salts up to 20%, and a large variety of raw materials.

Inorganic hydrocolloids such as magnesium aluminum silicate stabilize suspensions, perfect emulsions, and optimize flow properties. Salts, surfactants, and water-miscible solvents will increase their viscosity. Inorganic hydrocolloids are also often used synergistically with organic thickeners. These combinations allow viscosity properties beyond what is possible with either the clay or organic thickener alone.

Saturated fatty alcohols such as cetearyl alcohol, cetyl alcohol, and stearyl alcohol act as permeation enhancers. They are mainly distributed to the stratum corneum because of its lipophilicity and interact with the stratum corneum lipids. This effect results in a more rapid and sustained diffusion of the micellar-borne active agent molecules across the skin.

Moisturizers such as dimethicone, glycerin, and wheat germ oil are used to restore the barrier function of the epidermis and to cover tiny fissures in the skin. They provide a soothing protective film and increase the water-content of the epidermis. Emollient moisturizers such as dimethicone hydrate and improve the appearance of the skin by contributing to skin softness and enhanced flexibility. They serve to fill the cracks between clusters of desquamating corneocytes and are not usually occlusive unless applied heavily. Humectant moisturizers such as glycerin are able to attract water from the dermis and the external environment. They hasten the maturity of immature corneocytes into more resilient and protective cells as they migrate through the epidermis. Occlusive moisturizers such as wheat germ oil reduce trans-epidermal water loss by creating a hydrophobic barrier over the skin and contributing to the matrix between corneocytes. Their main limitations include odor and a greasy feel.

LO is an organic mixture that supports mold growth. Anti-microbial preservatives are often added to it in order to supplement intrinsic anti-microbial activity. The development of LO with adequate anti-microbial activity may prevent the problems that could occur from microbial contamination or proliferation during storage. The United States Pharmacopeia (USP) details guidance on the performance and interpretation of preservative efficacy testing.

Euxyl® PE9010 (Schulke & Mayr, Norderstedt, Germany) (Ethylhexylglycerin, Phenoxyethanol) is a paraben-free preservative. It is a multifunctional additive that enhances the efficacy of phenoxyethanol. The addition of ethylhexylglycerin affects the interfacial tension at the cell membrane of microorganisms and improves the preservative activity of phenoxyethanol. It is a complete broad spectrum antimicrobial preservative system that is effective against Gram-positive and Gram-negative bacteria, yeast, and mold.

EDTA is used as a chelating agent. It binds minerals which are necessary components for the growth of mold. EDTA also pulls heavy metals from your skin upon application.

Preferred embodiments of the invention may include lecithin organogel compositions which provide high penetrating power, which are time-efficient and uniform, which have improved stability, which have a high uptake capacity for ionic active drugs, and which are cosmetically elegant and mold resistant.

### DETAILED DESCRIPTION OF PREFERRED EMBODIMENTS

One aspect of the current invention pertains to a lecithin organogel composition which may be used to deliver pharmaceutical products transdermally. The invention further comprises a method for producing the lecithin organogel composition, which may contain up to 80% additive ingredients. Preferred embodiments of the invention may include lecithin organogel compositions which provide high penetrating power, which are time-efficient and uniform, which have improved stability, which have a high uptake capacity for ionic active drugs, and which are cosmetically elegant and mold resistant.

A preferred embodiment of the lecithin organogel composition comprises one or more non-ionic oil-in-water (“O/W”) emulsifier agents, non-ionic water-in-oil (“W/O”) emulsifier agents, inorganic hydrocolloids, organic hydrocolloids, biocompatible surfactants, nonpolar solvents, saturated fatty alcohols, moisturizers, preservatives/antimicrobials, chelating agents, and various mixtures thereof.

A preferred embodiment of the lecithin organogel composition comprises a non-ionic oil-in-water (O/W) emulsifier agent with a hydrophile-lipophile balance (“HLB”) value from about 8 to about 18. HLB is calculated by the formula:

HLB=20(1−S/A)

wherein S=saponification number of the ester
A=acid number of the recovered acid

O/W emulsifiers include the group consisting of polyethoxylated emulsifiers and polypropoxylated emulsifiers. Examples may include ceteth-15, ceteth-16, ceteth-20, ceteareth-6, ceteareth-12, ceteareth-15, ceteareth-16, ceteareth-20, ceteareth-25, isoceteth-20, isosteareth-20, steareth-10, steareth-20, oleth-5, oleth-10, oleth-15, oleth-20, laureth-15, PEG-20 stearate, PEG-25 stearate, PEG-20 oleate, PEG-20 sorbitan stearate, PEG-20 sorbitan isostearate, PEG-20 sorbitan oleate, sodium laureth-11 carboxylate, sodium lauryl ether sulfate, PEG-30 cholesteryl ether, PEG-60 evening primrose glyceride, bis PEG/PPG-16/16 PEG/PPG16/16 dimethicone+caprylic/capric triglyceride, PEG-45 palm kernel oil glyceride, PEG-20 glyceryl laurate, PEG-20 glyceryl stearate, and PEG-20 glycerol isostearate. The non-ionic O/W emulsifying agent may be present in a concentration range of 0. 1% to 4.0%, preferably 0.5% to 3.0%, most preferably 1.0% to 2.0%. One preferred example is Lipowax® D (Lipo Chemicals Inc., Patterson, N.J.), a combination of cetearyl alcohol and ceteareth-20.

This embodiment of the invention further comprises a non-ionic water-in-oil (W/O) emulsifier agent with a hydrophile-lipophile balance (“HLB”) value from about 1 to about 8. W/O emulsifiers include the group consisting of esters of alkanecarboxylic acid emulsifiers. Examples may include glyceryl caprinate, glyceryl caprylate, glyceryl dilaurate, glyceryl laurate, glyceryl linoleate, glyceryl oleate, glyceryl ricinoleate, glyceryl stearate, glycerol isostearate, diglycerol isostearate, triglycerol diisostearate, sorbitan isostearate, propylene glycol isostearate, propylene glycol stearate, polyglyceryl-3 methylglucose distearate, methylglucose sesquistearate, and polyglyceryl-2 dipolyhydroxystearate. The non-ionic W/O emulsifying agent may be present in a concentration range of 0.1% to 4.0%, preferably 0.5% to 3.0%, most preferably 1.0% to 2.0%. A preferred example is Emulsynt® GDL (ISP, Wayne, N.J.), which is glyceryl dilaurate.

This embodiment of the invention further comprises an inorganic hydrocolloid with modified or unmodified, naturally occurring or synthetic sheet silicates. Inorganic hydrocolloids include the group consisting of natural and synthetic bentonites, hectorites, and hydrotalcites. Examples may include bentonites such as magnesium aluminum silicate, quaternium-18 bentonite and stearalkonium bentonite, hectorites such as sodium magnesium silicate, quaternium-18 hectorite and stearalkonium hectorite, and hydrotalcites such as magnesium aluminum silicate synthesized with long-chain, organic and ammonium salts. The inorganic hydrocolloid may be present in a concentration range of 0.5% to 5.0%, preferably 1.0% to 4.0%, most preferably 2.0% to 3.0%. A preferred example is Veegum® HS (RT Vanderbilt Company, Norwalk, Conn.), which is magnesium aluminum silicate.

This embodiment further comprises an organic hydrocolloid with long-chain, straight or branched polysaccharides that contain hydroxyl groups that can bond to water molecules. Organic hydrocolloids include the group consisting of natural and synthetic gums, polymers, and starches. Examples may include gums such as gum arabic, gum karaya, gum tragacanth, gum ghatti, agar-agar, guar gum, locust bean gum, konjac, alginates, carrageenans, pectin, tara gum, xanthan gum, gellan gum, pullulan, curdlan, cellulose, microcrystalline cellulose, carboxymethylcellulose gum, methylcellulose, hydroxypropylcellulose, gelatin and chitosan, polymers such as acrylates/alkyl acrylate copolymer, acrylates/alkyl acrylate crosspolymer, acryloyldimethyltaurate copolymer and acryloyldimethyltaurate crosspolymer, and starches such as hydroxypropyl starch phosphate. The organic hydrocolloid may be present in a concentration range of 1.0% to 6.0%, preferably 2.0% to 5.0%, most preferably 3.0% to 4.0%. A preferred example is the starch Structure® XL (National Starch, Bridgewater, N.J.).

This embodiment further comprises a biocompatible surfactant with phosphatidylcholine. Biocompatible surfactants include naturally occurring unsaturated lecithins. Examples may include soy bean lecithin and egg yolk lecithin. The biocompatible surfactant may be present in a concentration range of 1.0% to 6.0%, preferably 2.0% to 5.0%, most preferably 3.0% to 4.0%.

This embodiment further comprises a nonpolar solvent with an ability to form gel in the presence of lecithin. Nonpolar solvents include alkanes, esters and amines. Examples may include alkanes such as cyclopentane, cyclooctane, trans-decalin, trans-pinane, n-pentane, n-hexane and n-hexadecane, esters such as ethyl laureate, ethyl myristate, isopropyl myristate and isopropyl palmitate, and amines such as tripropylamine. The nonpolar solvent may be present in a concentration range of 2.0% to 12.0%, preferably 4.0% to 10.0%, most preferably 6.0% to 8.0%. A preferred example is isopropyl palmitate.

If desired, a saturated fatty alcohol such as myristyl alcohol, pentadecanol, cetyl alcohol, cetearyl alcohol, stearyl alcohol, nonadecanol, arachidyl alcohol, heneicosanol, behenyl alcohol, brassidyl alcohol, lignoceryl alcohol, ceryl alcohol and myricyl alcohol may be used in the present invention. The fatty alcohol has the ability to provide a transitory effect on membrane permeability. The saturated fatty alcohol may be present in a concentration range of 0.1% to 4.0%, preferably 0.5% to 3.0%, most preferably 1.0% to 2.0%. Preferred examples include cetyl alcohol and stearyl alcohol.

If desired, a moisturizer such as dimethicone, glycerin and wheat germ oil may be used in the present invention. The moisturizer stabilizes the skin prior to transmigration of the active agent and assists the skin to repair any damage. The moisturizer may be present in a concentration of 0.2% to 1.2%, preferably 0.4% to 1.0%, most preferably 0.6% to 0.8%. In a preferred example, a mixture of dimethicone, glycerin, and wheat germ oil is used.

If desired, a preservative or an antimicrobial agent such as Euxyl® PE9010 (Schulke & Mayr, Norderstedt, Germany) (Ethylhexylglycerin, Phenoxyethanol) may be included in the present invention. The antimicrobial agent is equally effective against bacteria, yeasts and mold fungi. The antimicrobial agent may be present in a concentration of 0.5% to 1.0%, preferably 0.6% to 0.9%, most preferably 0.7% to 0.8%.

If desired, a chelating agent such as EDTA may be included in the present invention. The chelating agent sequesters di- and trivalent metal ions. The chelating agent may be present in a concentration of 0.05% to 0.10%, preferably 0.06% to 0.09%, most preferably 0.07% to 0.08%.

The lecithin organogel (“LO”) composition may be prepared by blending the proper amounts and ratios of all the required ingredients together. This LO can later be used to dissolve active drugs to make the final prescription gel composition.

One embodiment of the invention would include preparation as follows:

Oil phase: A stainless steel tank is charged with Lipowax® D (Lipo Chemicals Inc., Patterson, N.J.), Cetyl Alcohol, Stearyl Alcohol, Emulsynt® GDL (ISP, Wayne, N.J.), and Wheat Germ Oil are added. The mixture is heated to 75-80° C. Mixing is carried out for 30 minutes or until homogenous.

Inorganic hydrocolloid phase: A stainless steel tank is charged with purified water and heated to 35-40° C. Veegum® HS (RT Vanderbilt Company, Norwalk, Conn.) is then added and mixed for 2 hours or until homogenous.

Organic hydrocolloid phase: A double-motion kettle is charged with purified water and heated to 35-40° C. The mixer/sweeper is turned to 60 Hz. Structure® XL (National Starch, Bridgewater, N.J.) is added and the mixer/sweeper is increased to 90 Hz. Mixing and sweeping is carried out for 2 hours or until homogenous.

Emulsion phase: The double-motion kettle mixer/sweeper is turned to 60 Hz. The inorganic hydrocolloid phase is added to the organic hydrocolloid phase. Glycerin and EDTA are then added. The mixer/sweeper is increased to 90 Hz. Mixing and sweeping is carried out for 30 minutes or until homogenous. Then the mixer/sweeper is decreased to 60 Hz and heated to 75-80° C. The oil phase is added and the temperature is maintained at 75-80° C. The mixer/sweeper is increased to 90 Hz, and mixing and sweeping continues for 20 minutes or until homogenous. Then the mixer/sweeper is decreased to 60 Hz and cooled to 35-40° C. Lecithin 33% Solution is then added and mixing and sweeping continues for 20 minutes or until homogenous. The mixer/sweeper is decreased to 30 Hz and cooled to 25-30° C. Euxyl® PE9010 (Schulke & Mayr, Norderstedt, Germany) is then added, with Dow Corning (Midland, Mich.) 200-350. Mixing and sweeping continues for 20 minutes or until homogenous.

### EXAMPLE 1

Table 1 below shows an example of one embodiment, including preferred ingredients and amounts.

TABLE 1

| Ingredient | Amount |
|---|---|
| Purified Water | 76.65 w/w |
| Veegum ® HS | 1.75 w/w |
| Structure ® XL | 6.00 w/w |
| Glycerin | 0.75 w/w |
| EDTA | 0.05 w/w |
| Lipowax ® D | 2.25 w/w |
| Cetyl Alcohol | 0.75 w/w |
| Stearyl Alcohol | 0.75 w/w |
| Emulsynt ® GDL | 0.50 w/w |
| Wheat Germ Oil | 0.50 w/w |
| Lecithin | 3.00 w/w |
| Isopropyl Palmitate | 6.00 w/w |
| Euxyl ® PE9010 | 1.00 w/w |
| Dow Corning 200-350 | 0.05 w/w |

This embodiment of the invention was prepared as follows:

To prepare the oil phase, a stainless steel tank was charged with Lipowax® D (Lipo Chemicals Inc., Patterson, N.J.). Cetyl alcohol, stearyl alcohol, Emulsynt® GDL (ISP, Wayne, N.J.), and Wheat Germ Oil were added. The temperature was raised to 75-80° C., and mixing was carried out for about 30 minutes or until homogenous.

To prepare the inorganic hydrocolloid phase, a stainless steel tank was charged with purified water and heated to 35-40° C. Veegum® HS (RT Vanderbilt Company, Norwalk, Conn.) was added and mixed for about 2 hours or until homogenous.

To prepare the organic hydrocolloid phase, a double-motion kettle was charged with purified water and heated to 35-40° C. The mixer/sweeper was turned on to 60 Hz. Structure® XL (National Starch, Bridgewater, N.J.) was added and the mixer/sweeper was increased to 90 Hz. Mixing and sweeping were carried out for 2 hours or until homogenous.

To prepare the emulsion phase, the double-motion kettle mixer/sweeper was turned on to 60 Hz. The organic hydrocolloid phase was added to the organic hydrocolloid phase, followed by glycerin and EDTA. The mixer/sweeper was increased to 90 Hz and mixing and sweeping were carried out for 30 minutes or until homogenous. The mixer/sweeper was then decreased to 60 Hz and heated to 75-80° C. Then the oil phase was added, while maintaining the temperature at 75-80° C. The mixer/sweeper was increased to 90 Hz. Mix and sweeping continued for about 20 minutes or until homogenous. The mixer/sweeper was then decreased to 60 Hz and cooled to 35-40° C. Lecithin 33% Solution was added (in isopropyl palmitate), and mixing and sweeping were carried out for about 20 minutes or until homogenous. Then the mixer/sweeper was decreased to 30 Hz and cooled to 25-30° C. Euxyl® PE9010 (Schulke & Mayr, Norderstedt, Germany) was then added, with Dow Corning 200-350. Mixing and sweeping continued for 20 minutes or until homogenous.

### REFERENCES CITED

### U.S. Patent Documents

U.S. Pat. No. 5,654,337

U.S. Pat. No. 5,716,639

U.S. Pat. No. 5,837,289

U.S. Pat. No. 6,290,986

### Other Publications

Lawrence M. J., 2000

Crandall W. T., 2001

Jibry N., 2004

Willimann H., 1992

Brucks R., 1998

What is claimed is:

1. A lecithin organogel composition comprising:
non-ionic oil-in-water (“O/W”) emulsifier agent having a hydrophile-lipophile balance (“HLB”) ranging from about 8 to about 18;
non-ionic water-in-oil (“W/O”) emulsifier agent having a hydrophile-lipophile balance (“HLB”) ranging from about 1 to about 8;
inorganic hydrocolloid;
organic hydrocolloid;
biocompatible surfactant;
nonpolar solvent;
saturated fatty alcohol;
moisturizer;
preservative;
chelating agent; and
mixtures thereof

2. The lecithin organogel composition of claim 1, wherein the non-ionic oil-in-water (“O/W”) emulsifier agent is ceteth-15, ceteth-16, ceteth-20, ceteareth-6, ceteareth-12, ceteareth-15, ceteareth-16, ceteareth-20, ceteareth-25, isoceteth-20, isosteareth-20, steareth-10, steareth-20, oleth-5, oleth-10, oleth-15, oleth-20, laureth-15, PEG-20 stearate, PEG-25 stearate, PEG-20 oleate, PEG-20 sorbitan stearate, PEG-20 sorbitan isostearate, PEG-20 sorbitan oleate, sodium laureth-11 carboxylate, sodium lauryl ether sulfate, PEG-30 cholesteryl ether, PEG-60 evening primrose glyceride, bis PEG/PPG-16/16 PEG/PPG 16/16 dimethicone+caprylic/capric triglyceride, PEG-45 palm kernel oil glyceride, PEG-20 glyceryl laurate, PEG-20 glyceryl stearate, PEG-20 glycerol isostearate, and mixtures thereof.

3. The lecithin organogel composition of claim 1, wherein the non-ionic water-in-oil (“W/O”) emulsifier agent is glyceryl caprinate, glyceryl caprylate, glyceryl dilaurate, glyceryl laurate, glyceryl linoleate, glyceryl oleate, glyceryl ricinoleate, glyceryl stearate, glycerol isostearate, diglycerol isostearate, triglycerol diisostearate, sorbitan isostearate, propylene glycol isostearate, propylene glycol stearate, polyglyceryl-3 methylglucose distearate, methylglucose sesquistearate, polyglyceryl-2 dipolyhydroxystearate, or mixtures thereof.

4. The lecithin organogel composition of claim 1, wherein the inorganic hydrocolloid is magnesium aluminum silicate, quaternium-18 bentonite, stearalkonium bentonite, sodium magnesium silicate, quaternium-18 hectorite, stearalkonium hectorite, magnesium aluminum silicate with ammonium salts, or mixtures thereof.

5. The lecithin organogel composition of claim 1, wherein the organic hydrocolloid is gum arabic, gum karaya, gum tragacanth, gum ghatti, agar-agar, guar gum, locust bean gum, konjac, alginates, carrageenans, pectin, tara gum, xanthan gum, gellan gum, pullulan, curdlan, cellulose, microcrystalline cellulose, carboxymethylcellulose gum, methylcellulose, hydroxypropylcellulose, gelatin and chitosan, polymers such as acrylates/alkyl acrylate copolymer, acrylates/alkyl acrylate crosspolymer, acryloyldimethyltaurate copolymer, acryloyldimethyltaurate crosspolymer, hydroxypropyl starch phosphate, or mixtures thereof.

6. The lecithin organogel composition of claim 1, wherein the biocompatible surfactant is a naturally occurring unsaturated lecithin, or mixtures thereof.

7. The lecithin organogel composition of claim 1, wherein the nonpolar solvent is isopropyl palmitate.

8. The lecithin organogel composition of claim 1, wherein the saturated fatty alcohol is myristyl alcohol, pentadecanol, cetyl alcohol, cetearyl alcohol, stearyl alcohol, nonadecanol, arachidyl alcohol, heneicosanol, behenyl alcohol, brassidyl alcohol, lignoceryl alcohol, ceryl alcohol, myricyl alcohol, or mixtures thereof.

9. The lecithin organogel composition of claim 1, wherein the moisturizer is dimethicone, glycerin, wheat germ oil, or mixtures thereof.

10. The lecithin organogel composition of claim 1, wherein the preservative is a mixture of ethylhexylglycerin and phenoxyethanol.

11. The lecithin organogel composition of claim 1, wherein the chelating agent is EDTA.
